(19) United States
(12) Patent Application Publication
(10) Pub. No.: US 2005/0288797 A1
(43) Pub. Date: Dec. 29, 2005

(54) Controlled absorption biograft material for autologous tissue support

(75) Inventor: Charles A. Howland, Temple, NH (US)

Correspondence Address: MAINE & ASMUS, 100 MAIN STREET, P O BOX 3445, NASHUA, NH, 03061-3445, US

(73) Assignee: WARWICK MILLS, INC., New Ipswich, NH (US)

(21) Appl. No.: 11/165,453

(22) Filed: Jun. 23, 2005

Related U.S. Application Data
(60) Provisional application No. 60/582,373, filed on Jun. 23, 2004.

(51) Int. Cl. A61F 2/02

(52) U.S. Cl. 623/23.74; 606/151; 435/399; 623/1.51

(57) ABSTRACT

An implantable tissue grafting medical system and material using a combination of bio-absorbable and non bio-absorbable fibers and materials such as Poly Glycolic Acid (PGA) and polyester (PET), provides a permeable mesh or weave of fibers with an initial interstice size and permeability factor suitable to initial implant requirements, and a pre-engineered bio-absorption pattern and rate that controls the gradual expansion of interstice size within the mesh or weave in one or two dimensions up to a pre-engineered maximum interstice size, consistent with the anticipated rate of tissue regeneration on the implant, while retaining a primary grid or circumferential pattern of non-absorbable fibers at the maximum interstice size for supporting the new tissue for an extended period. Various means for combining materials to obtain initial interstice size, pattern and permeability, with the desired absorption pattern and rate, and the desired end point interstice size and spacing, are also disclosed.

This application relates to and claims priority to pending U.S. application Ser. No. 60/582,373, filed Jun. 23, 2004.

### BACKGROUND OF THE INVENTION

1. Technical Field of the Invention

This invention most generally relates to a surgical implantation material. More particularly, the invention relates to a controlled absorption biograft material for autologous tissue support.

2. Background Art

Grafts for surgical implantation have been manufactured of Dacron or other PET (polyester) fibers. These knit or woven structures generally are produced of 20-200 denier multifilament yarns. Depending on the application the permeability (perm) of these materials is moderate to low. For reconstructive layers a perm of 100-600 cfm/ft2 is representative. In the vascular area the perms are lower for the retention of red blood and other serum components. In this area perm in the order of 0-100 cfm/ft2 is representative. In addition to these basic structural characteristics, PET materials are generally inert and are little affected by hydrolysis and oxidative attack in the body. Collagen tissue in growth in these materials is well known, for example in heart valve sewing rings in most cases, thrombus with the adjacent myocardium, or aortic wall covers the textile surface over some time period after implantation. This overgrowth should optimally yield a thin layer of largely endeothielized mature-collagen. However the pathology of the tissue interaction with PET is not constructive to the operation of the graft materials. The various tissue types that over grow the PET surface are not required for graft performance. In fact there is a negative impact from tissue over growth over-colonization of fibroblasts which leads to scar formation, calcification, and necrosis can affect the competence of the graft.

### SUMMARY OF THE INVENTION

The invention concerns an implantable tissue grafting medical system and process, including materials and sub processes, using a combination of bio-absorbable and non bio-absorbable fibers and materials, examples of which include Poly Glycolic Acid (PGA) and polyester (PET). The materials of the invention are combined and constructed in some embodiments as a permeable mesh structure or weave of fibers with an initial interstice size and permeability factor suitable to initial implant requirements. The structure or weave of fibers has a pre-engineered bio-absorption pattern and absorption rate profile that controls the gradual expansion of interstice size within the mesh or weave in one or two dimensions up to a pre-engineered maximum interstice size, at a rate consistent with the anticipated rate of tissue regeneration on the implant. It does this while retaining a primary grid or circumferential pattern of non-absorbable fibers at the maximum interstice size calculated for supporting the new tissue for an extended period. Various means are described for combining the materials of the invention to obtain initial interstice size, pattern and permeability, with the desired absorption pattern and rate, and the desired end point interstice size and spacing.

A primary object of the invention is to provide an implantable medical material having controlled bio-absorption rate.

Another object of the invention is to provide an implantable medical material using at least one bio-absorbable fiber and at least one non-bio-absorbable fiber to form a mesh for cell colonization.

The claimed invention discloses in one respect an implantable medical material having bio-absorbable materials of at least two different absorption rates. The different bio-absorbable material fibers are woven together so as to provide a pattern of many initially small interstices, with a controlled rate and pattern of absorption of the implantable medical material that combines and enlarges the remaining interstices in an ordered progression of pattern enlargement until all the bio-absorbable materials are absorbed.

The claimed invention discloses in another respect an implantable medical material having at least one bio-absorbable material fiber and at least one non-bio-absorbable material fiber. The bio-absorbable material fibers and the non-bio-absorbable material fibers are prepared for weaving and then woven together to form an implantable medical device that behaves when implanted in such a manner that the combination of the bio-absorbable material fibers and the non-bio-absorbable material fibers controls the rate of absorption of the bio-absorbable portion of the implanted medical material, so that the original multiple, small interstices of the woven device are gradually combined and enlarged by absorption at about the rate of new tissue growth until only a final pattern of fewer, larger interstices formed by the remaining non-absorbable materials remains.

The claimed invention also discloses an implantable medical material having a first weaving direction and a second weaving direction. The implantable medical material has at least one bio-absorbable material fiber running in the first weaving direction and at least one non-bio-absorbable material fiber running in both first and second weaving directions. The bio-absorbable material fibers and the non-bio-absorbable material fibers are woven together, and the bio-absorbable material fibers are interspersed with the non-absorbable material fibers in the first weaving direction.

Various techniques are disclosed that may be applied to the bio-absorbable materials to affect the actual rate of absorption of specific fibers or fiber bundles, which by selected placement within the mesh or weave, controls the overall absorption profile of the material.

Still other objects and advantages of the present invention will become readily apparent to those skilled in this art from the following detailed description, wherein I have shown and described several embodiments of the invention, simply by way of illustration of the best mode contemplated by me on carrying out my invention. As will be realized, the invention is capable of other and different embodiments, and its several details are capable of modifications in various obvious respects, all without departing from the invention.

### BRIEF DESCRIPTION OF THE DRAWINGS

FIG. 1 is an illustration of the gradient of absorption of a patch graft within autologous tissue.

FIG. 2 is an illustration of a first embodiment material of the invention having a repeating pattern of one dimensional absorption gradient frames, using absorbable materials of two rates.

FIG. 3 is an illustration of phase one early stage cell colonization in the embodiment of FIG. 2.

FIG. 4 is an illustration of phase two later stage cell colonization in the embodiment of FIG. 2, with the absorbable fiber in a degraded state.

FIG. 5 is an illustration of phase three yet later stage cell colonization in the embodiment of FIG. 2, with the absorbable fiber nearly completed absorbed and the cell colonization extended to encapsulate the non-absorbable fibers.

FIG. 6 is an illustration of a second embodiment material of the invention having a repeating pattern of two dimensional absorbable gradient frames utilizing absorbable fibers of different rates to affect the pattern of absorption and frame center expansion.

FIG. 7 is an illustration of the embodiment of FIG. 6 showing the early stage effects of controlled expansion of the frame center.

FIG. 8 is an illustration of the embodiment of FIG. 6 showing the later stage effects of controlled expansion of the frame center.

### DESCRIPTION OF THE PREFERRED EMBODIMENT

This invention substitutes some of the inert fiber in graft structures with bio-absorbable fiber. The woven structure is woven so that absorption rates of bio-absorbable material approximate the tissue regeneration rate during the post-operative healing process. Using the example of a vascular graft, these two processes operate as follows. The endothelial and fibroblasts begin to populate the surface of the graft. This process is more pronounced at the interface with the autologous tissue. As the tissue remodeling takes place bio-absorbable fiber is being broken down, at an approximately matched rate of tissue remodeling, in the graft. As void spaces are created through interstice degradation, the autologous tissue populates these voids.

Because the regenerative process of tissue remodeling is slow, the structural integrity of the graft must be maintained by a second matrix of non-absorbable fiber. In the case of the vascular graft this second matrix can be of PET or other compatible non-absorbable fiber like Teflon or PEN (Polyethylene Naphthalate).

The bio-absorbable material and non-absorbable material need not be distributed in a symmetric way in both directions in the web. For example in a vessel the web would have a higher density if one or both the absorbable and non-absorbable fiber in the conferential direction as this direction is subjected to higher loading than the axial direction in the vascular or other vessel configuration. Over time only the non-absorbable or PET like material remains as an open mesh. In the ideal case healing and remodeling encapsulate the non-absorbable fiber with a continuous layer of differentiated collagen tissue. As the tissue remodeling proceeds, and the graft is absorbed, active stresses are transferred to newly generated cell tissue which perpetuates the tissue remodeling process into the advanced stages.

Absorption gradients in two (2) dimensions refers to the variable rate at which the substrate material is absorbed, or degrades. In a woven structure the term “two dimensional” refers to absorption in both the X and Y directions. In a woven this would correspond to warp and fill directions, and in a knit or non woven this would correspond to the Cross Machine Direction (CMD) and the Machine Direction (MD). Having gradients of absorption in graft structures allows for larger zones of remodeling tissue between non-absorbed fiber supports. The slow growth rates of the remodeling tissue are accommodated by 2 or more rates of decomposition of absorbed fiber, which together provide an extended or graduated graph of available support structure declining over time.

If the frame or zone of remodeled tissue requires more time than is allowed by a single decomposition rate the remodeling tissue must take on stress before it is competent. In this invention the larger zones or frames of tissue can be grown by designing in more than one rate of decomposition in the graft. Generally, in tissue remodeling cell growth is more rapid nearest the contact points between graft and autologous tissue than it is at a 5-10 mm distance away from these contact points. In order to promote full penetration of the graft, absorption needs to be matched as closely as possible to these varying rates of growth. By building a gradient into the graft the absorption rates of bio-absorbable material and the various rates of tissue growth can be matched more closely.

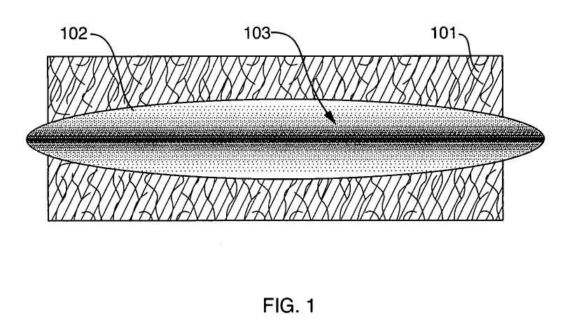

Referring to FIG. 1, which is an illustration of the gradient 103 of absorption of a patch graft 102 within autologous tissue 101. By way of explanation, the gradient is shown in one direction. In practice both single and dual direction gradients 103 are useful. In the case of a more rectangular patch graft 102 the contact points are on both top and sides of the graft 102 and a dual gradient device would be preferable for this structure. In attachment to vessels the single direction gradient 103 is more common.

This design allows that not all areas of a graft structure will remodel and absorb at a balanced rate. For example, sewing rings for heart valves, re-endothilialization of bio-prosthesic valves is slow and limited to basal attachments and can remain incomplete for long periods after implantation. Understanding that the process by which tissue remodels itself is not consistent throughout the area of implementation reinforces the importance of creating a structure with differential gradients of absorption. A graft with gradients of absorption allows for the most complete and natural course of remodeling.

The relative value of the bio-absorbable fiber is dependent on the interaction with healing mechanisms in the body. In addition to the types of stresses, the time frame for healing will suggest the optimum fiber type for use in a structure. That having been said, Poly Glycolic Acid (PGA) is the preferred bio-absorbable fiber. PGA has been used for many year as a suture material and has well understood biocompatibility and absorption rates that are consistent with surgical healing process. PGA has good tensile strength and small denier per filament size and is readily processed with fiber spinning, twisting, braiding, knitting, weaving and other non-woven fiber processing equipment. In most vessel related examples the likely to range of denier is 10-300 as most favorable for perm and structural requirements. PGA fiber has also been shown to be a material that is capable of hosting cellular migrations in live animal studies.

Determining techniques for creating controlled gradients of absorption in the bio-absorbable material is the first step in the assembly process of the substrate. The rate of absorption for fibers will be dependent on site of use, which will dictate stress factors and time requirements of fiber performance. The following examples are preferred techniques for creating gradients of absorption, which can be used, independently or combined, to meet requirements with regards to differential absorption rates of tissue regeneration.

Differentiating denier selection allows for controlled absorption rates by virtue of the fact that bio-absorbable materials degrade, all things being equal, at constant rates. Therefore, the selection of a higher denier translates into more bio-absorbable material in the cross-section and, all things being equal, a longer total time to decomposition. However, tissue remodeling does not happen at a constant rate throughout the area being remodeled. During tissue remodeling there may different absorption requirements that must be met, within the same structure, in order to support the autologous tissue remodeling. Those requirements are largely dependent on the application and location of the graft within the body. Once the requirements for absorption have been determined, denier selection and placement in both warp and fill, or only warp or fill, can be selected accordingly. Choosing different denier sizes and incorporating them into the woven structure results in a woven product or device that possesses a gradient, or varied rate, of absorption, in one or both directions. Essentially, due to the nature of the weaving process, denier differentiation allows for an absorption gradient that can be engineered to vary or differ interstice by interstice.

The braiding of fibers is also a technique that can be used to create controlled absorption rates of biomaterial in the tissue remodeling process. The absorption rate of braided ends can be adjusted by adding or limiting the number of fibers that will be included in a braided end. The more ends incorporated into a braid, the slower the absorption rate. Inversely, the fewer ends incorporated into a braid, the more rapidly it is absorbed. By using the braiding method for constructing the yarns or ends of a woven, both warp and fill thickness of the braided ends can be varied to the degree that every braided end can be constructed to be of different thickness, and thus all ends would possess different absorption rates. Since all braided ends could be constructed of different absorption rates, braiding also provides a means to creating a woven structure with gradients of absorption. Once location and absorption requirements are determined, braided ends can be produced and woven or assembled in selected warp and fill positions so that the two-dimensional absorption gradient profile of the finished structure matches these requirements.

Twisting also allows for differential, yet controlled, absorption rates. By twisting fiber there are areas of a fiber strand that do not get exposed to degrading factors directly due to the inter-fiber contact area. These protected blind spots limit the exposed surface area of the bio-absorbable fibers, resulting from the twisting process, slow the absorption rate. Absorption rates can be controlled then, on an end by end basis, by the number of twists per inch that are applied to a particular end. The twisting method may also be combined with absorption process controls such as denier size differentiation or braiding. Plying two different denier sizes, braided ends, or twisted ends together is another control process for absorption rates.

The principle behind the use of bio-absorbable material is to provide a conducive, yet temporary, environment for cell colonization which leads to tissue remodeling. In order to do this a bio-absorbable material must promote growth of cells by providing initial colonization areas or sites for endothelial cell growth. As endothelial cells populate and lay the groundwork for the presence of collagen the bio-absorbable material must give way, at a calculated rate in accordance with the invention, so that the endothelial cells and collagen are forced to accept stress loads, which in turn initiates an expansive growth response which leads to autologous tissue structures. Proper functioning of the bio-absorbable material is critical to autologous tissue remodeling. Because tissue remodeling requires varied performances of bio-absorbable material inside the same substrate structure, it is equally critical that these varied zones of performance within a given substrate remain in the position they were initially designed to function in. One such solution to this requirement to stop location shifting of bio-absorbable material is to use a leno lock or work knit lock in the inert material ends. The locking of inert fiber at the looping crossing points provides a non-raveling edge or boundary in the substrate to keep the bio-absorbable materials in place at the edge of graft. This technique can be applied to graft structures like a valve leaflet that would otherwise have a free edge that is undesirable as a suturing anchor point at implantation.

Upon weaving with a leno lock in the inert material the finished woven can then be heat treated to an appropriate temperature. This heat treatment heats up the inert material (PET fiber) and in doing so creates a welding action at the contact or crossing point of the fibers. This results in a welded joint at crossing points of the PET fiber, while leaving the bio-absorbable material unaltered in the weave. The welding of inert fiber crossing points provides a rigid boundary within the surface plane of the substrate to keep the bio-absorbable materials constrained to designated areas.

The use of cover coatings on bio-absorbable materials provides another mechanism for control over absorption rates of bio-absorbable material in the tissue remodeling process. Coatings allow for a controlled absorption by introducing a process of absorption that involves stages of absorption. Coatings themselves vary in absorption rates. Casin, callogin, various gels and polymers can be used. The use of various coatings on bio-absorbable material offer more control over the absorption rates of bio-absorbable material. In applications that require limited size, or amounts, of denier, braids, or plying, yet demand slow absorption rates, coatings may be useful or necessary. In the case where a stent or other metallic element must bear on a graft at a contact point these coating can be applied locally to protect the graft and control the interface.

One way that a coating can be applied to bio-absorbable material is by means of the saturation method. For example, a PGA solution at five percent solids may be used as a cover coat. The bio-absorbable material is coated with the PGA solution by using the saturation method. The PGA coating can be applied to a woven substrate, or the coating may be applied to bio-absorbable material before it is woven. Also, the use multiple coats of one or more selected coating solutions in order to obtain a specific absorption characteristic is within the scope of the invention. With the option to coat bio-absorbable fiber before it is woven, after it is woven, as well as using multiple coats, specific gradients of absorption may be readily obtained to match the requirements of a particular tissue remodeling process.

In one embodiment, a woven sheet in the pattern of a plain weave consisting of PET fibers and bio-absorbable PGA is woven with the use of multi-beam warp and a rapier needle insertion loom. By using multiple beams in the warp, the degradation rate of the bio-absorbable material in the warp can be variably controlled from one warp end to another across the warp direction. The absorption and degradation rate of weft (or fill) insertions can also be controlled by loading bio-absorbable materials with different degradation and absorption rates. Thus, the bio-absorbable materials' degradation and absorption rate can be matched, interstice by interstice over its surface area, in accordance to tissue remodeling rates, which is dependent on the use, manipulation, transformation, and/or implementation site of the woven sheet in a graft structure.

For this setup, depending on requirements for above mentioned use and alterations, the warp feed is accomplished using multiple beams. Multiple beams allow for a controlled warp construction process that affords the advantages of different fiber types combinations, non-bio-active fibers and bio-active fibers, as well as combinations of bio-active materials that have different degradation rates. In weaving, fill insertion also allows for the use of multiple fibers. Constructing a bio-absorbable woven substrate that possesses a controlled gradient of absorption involves considering the following variables in conjunction with the afore mentioned weaving process.

Another embodiment of a woven structure, consisting of absorbable gradient frames, is constructed by using a combination of bio-absorbable material (PGA) and non-bio-absorbable material (PET) in warp and fill. Frames of absorption are constructed by combining bio-absorbable material with non-absorbable in either the warp, or fill, but not in both directions. In this embodiment, non-absorbable PET fiber and bio-absorbable PGA fiber combine to make the warp, which has an initial space of at least 0.75 mm between each warp end. In a pattern with denier differentiation, the bio-absorbable PGA(s) are inserted into the fill direction to create a woven structure of bio-absorbable and non-absorbable material with an initial permeability of less than 250 cfm. The end count for this embodiment is within the scope or range of 98-104 ends per inch. This variation stems from the implementation of one of the recited absorption rate control techniques used in bio-absorbable materials; in this case denier size differentiation. As described in the foregoing materials, viable techniques include one or a combination of twisting, braiding, denier size differentiation, cover coating, and plying of bio-absorbable material are being referenced. By using absorption control techniques in combination with the desired end count range, and weave patterns, the fibrous web of bio-absorbable and non-absorbable material is constructed to yield initial interstice sizes of less than 0.2 mm. The ideal permeation of the woven substrate is less than 250 cubic feet per minute (CFM) as verified by the Frazier Permeability test. This low level of permeation is necessary for the initial implementation of the substrate in order to perform as a competent conduit until tissue regeneration can begin.

As described above, one technique to create a gradient of absorption in the substrate is denier size differentiation. Preferred denier sizes for bio-absorbable material are in the 40 to 150 denier range. Submersing a woven structure that has been constructed using the specifications above, in plasma, for around 720 hours, will result in at least one filament losing around half its mass. Upon submersion of the same structure in plasma for around 4,320 hours, after the initial mass loss occurring during the first 720 hours, the mass retention, of at least one filament, is around 80% retention.

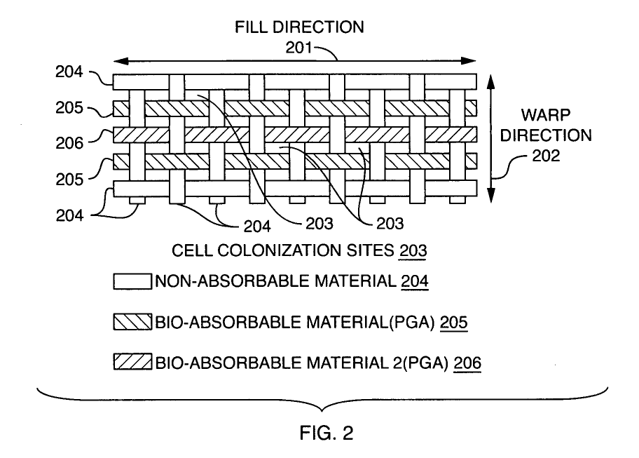

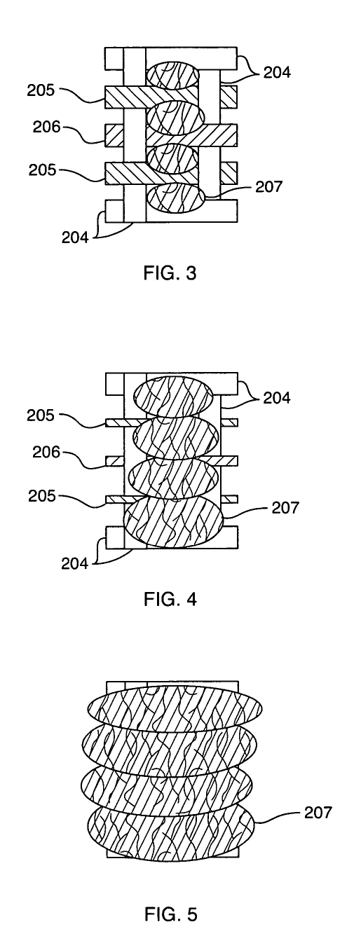

Referring now to FIG. 2, the bio-absorbable materials in this one dimensional example consists of fibers having one of two different rates of absorption. The first bio-absorbable material fibers 205 and the second bio-absorbable material fibers 206 both run in the fill direction 201, interspersed with non-absorbable material fibers 204, thus forming a repeating pattern of one dimensional absorption, gradient frames. This example displays a repeating pattern of bio-absorbable to non-absorbable material at a ration of 3 to 1. This ratio can be altered according to intended use of structure. The non-absorbable material fibers 204 also run in the warp direction 202, thus interlace with the bio-absorbable material fibers 205, 206 and non-absorbable material fibers 204 running in the fill direction 201, and form interstices. Initially small interstices provide sites 203 for cell colonization. As cells 207 grow, the bio-absorbable materials, for example PGA, are absorbed at their respective rates and cells 207 expand to reach, and later encapsulate, the non-absorbable material, for example PET, as is indicated in FIGS. 3, 4 and 5.

Denier size differentiation is used to obtain the different absorption rates of the bio-absorbable materials of fibers 205 and 206, but other techniques, including braiding, twisting, cover coating, and plying of the bio-absorbable material and fibers are also within the scope of the invention for producing fibers of differing absorption rates.

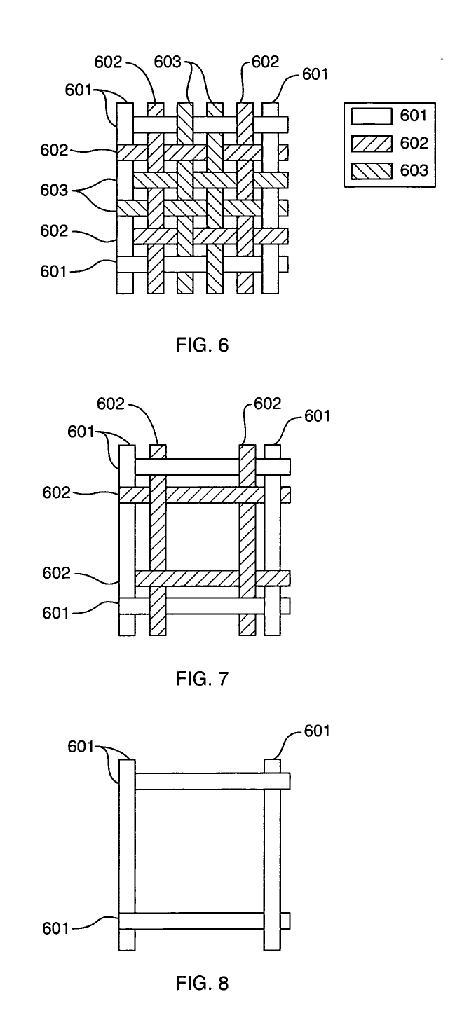

Referring now to FIGS. 6, 7 and 8, for this preferred embodiment of a woven structure, absorbable gradient frames are constructed by using a combination of bio-absorbable materials having multiple rates of absorption, and non-absorbable material, in warp and fill directions. Two dimensional absorption gradient frames are constructed by combining bio-absorbable material fibers 602 and 603, for example PGA, with non-absorbable material fibers 601, for example Dacron, in both the warp and fill directions.

In this preferred weave non-bio-absorbable material of Dacron along with bio-absorbable material PGA, of two different absorption rates, make up the pattern in the warp direction. In this embodiment, the first PGA fibers 602 have faster absorption rate than the second PGA fibers 603. Likewise, in a repeating pattern Dacron and bio-absorbable PGA are used in the fill direction. The resultant Dacron mesh or open frame limit is greater than 0.75 mm and preferably at least 1.5 mm across any given Dacron defined interstice. The preferred specifications for two dimensional absorption gradient frames are as follows. The end count should be within the scope of 98-104 ends per inch. This variation stems from the implementation of the absorption rate control techniques used in bio-absorbable materials. Specifically, braiding, twisting, denier size differentiation, cover coating, and plying of bio-absorbable material and fibers are among the techniques being referenced.

The preferred permeation of the complete woven substrate of non-absorbable material and absorbable material is less than 250 cubic feet per minute (CFM), as verified by the Frazier Permeability test. This low level of permeation is necessary for the initial implementation of the substrate in order for it to perform as a competent conduit or organ wall structure until tissue regeneration can begin.

The preferred technique to create a gradient of absorption in the substrate is denier size differentiating, although it is readily combined with the other cited techniques. Preferred denier size for bio-absorbable material is in the 40 and 150 denier range. The denier size results in an initial interstice size of 0.2 mm. The denier differentiation of absorption is engineered to produce a pattern where the fastest absorption takes place in the middle of the absorption gradient frame, as indicated by FIG. 7. The open frame center continues to expand with further absorption as indicated by FIG. 8. Absorption rates of PGA slow as the frame center expands towards the Dacron border. Under test conditions of submersing a woven structure that has been constructed using the specifications above, in plasma for around 720 hours, results in a loss of around half its mass for at least one filament. Upon submersion of the same structure in plasma for around 4,320 hours the mass retained, after the initial mass loss occurring during the first 720 hours, is 80%.

While the above embodiments have been described as using a combination of absorbable and non-absorbable materials, it will be appreciated that in all embodiments described, for select applications, the non-absorbable materials may be replaced with another class of relatively slowly absorbed materials that is simply less quickly, but never the less eventually, fully absorbed so that the device, while performing exactly as described until only the very slowly absorbed materials and the final grid or pattern they define remains, is then eventually full absorbed by the body over a further extended period of time. Accordingly, in the further embodiments described and the appended claims that follow, fibers recited as having relatively lower rates of bio-absorption may be further interpreted to include non-absorbable fibers.

The invention is susceptible of many more embodiments. For example, there is an implantable medical material consisting of a first pattern of multiple, relatively larger interstices or frames defined by first fibers (fibers having a first rate of absorption) of relatively lower rates of bio-absorption; and a second pattern of multiple, relatively smaller interstices within each frame of the first pattern defined by second fibers (fibers having a second rate of absorption) of relatively higher rates of bio-absorption in combination with the first fibers. The implantable medical material may include a third pattern of multiple, relatively yet smaller interstices within the first pattern, defined by third fibers (fibers having a third rate of absorption) of relatively yet higher rates of bio-absorption in combination with the first and said second fibers.

The fibers, preferably the second and third fibers, may be manufactured of Poly Glycolic Acid (PGA) or other bio-absorbable material. However the first fibers are more preferably manufactured from non-absorbable materials such as may be selected from among the group of materials consisting of Dacron, polyester (PET), Teflon and Polyethylene Naphthalate (PEN).

The second fibers may consist of fibers of at least two different absorption rates, where the rates are differentiated by at least one of the group of absorption rate differentiators or techniques consisting of using different denier sizes, braiding, twisting, plying, and coating of the bio-absorbable fibers. The second fibers may be made from the same materials as the third fibers but be similarly distinguished from the third fibers in their respective absorption rates by use of the same differentiators or techniques.

The size of the smallest interstices may be less than 0.2 mm in at least one dimension. The size of the relatively larger interstices formed by the first fibers may be at least 0.75 mm in at least one dimension. The permeability (prior to implanting the device) may be less than 250 cubic feet per minute (CFM) in accordance with the Frazier Permeability test. There may be a bio-compatible coating applied to the bio-absorbable material or fibers that reduces the respective rate of bio-absorption of selected fibers within the material.

The implantable medical material may consist of a woven material, where the first and second fibers are interspersed in at least one or both of warp and fill directions. The woven material may incorporate intersection locks of the first fibers at the free edges of the material, as by use of a leno lock or work knit lock or localized heat treatment or other method of securing or bonding a fiber intersection at or near the fiber ends.

The invention may also be embodied in methods and processes. For example, there is within the scope of the invention a method for making an implantable medical material comprising: combining first fibers (fibers having a first rate of bio-absorption) of relatively lower rates of bio-absorption with second fibers (fibers having a second rate of bio-absorption) of relatively higher rates of bio-absorption into an integrated pattern of multiple, relatively larger interstices or repetitive frames defined by the first fibers, and relatively smaller interstices or cell colonization sites within the relatively larger frames or interstices, where the smaller interstices are defined by the first and second fibers. The method may be extended to combining the first and second fibers with third fibers (fibers having a third rate of bio-absorption) of relatively yet higher rates of bio-absorption wherein said integrated pattern further includes relatively yet smaller interstices within said relatively larger interstices, said yet smaller interstices defined by said first, second and third fibers.

The method may extend to adjusting or engineering the rate of bio-absorption that will occur in the medical material by selecting and preparing at least some of the second and third fibers prior to their being combined, by using one or more criteria or technique selected from among the group of criteria or techniques consisting of denier sizing, braiding, twisting, plying, and coating of the fibers as has been discussed above. The method may likewise extend to fabrications of the material so as to achieve some or all of the preferred specifications previously described.

The objects and advantages of the invention may be further realized and attained by means of the instrumentalities and combinations particularly pointed out in the appended claims; where the words “fiber”, and “fibers” are to be interpreted broadly to include strands and threads and such, as well as bundles of fibers, strands and threads such as result from the disclosed techniques of twisting, braiding, and plying. Accordingly, the drawing and description are to be regarded as illustrative in nature, and not as restrictive.

1. An implantable medical material comprising
a first pattern of multiple, relatively larger interstices defined by first fibers of relatively lower rates of bio-absorption; and
a second pattern of multiple, relatively smaller interstices within said first pattern defined by second fibers of relatively higher rates of bio-absorption in combination with said first fibers.

2. The implantable medical material of claim 1, said first fibers comprising non-absorbable fibers.

3. The implantable medical material of claim 1, further comprising;
a third pattern of multiple, relatively yet smaller interstices within said first pattern defined by third fibers of relatively yet higher rates of bio-absorption in combination with said first and said second fibers.

4. The implantable medical material of claim 1, wherein said second fiber is manufactured of Poly Glycolic Acid (PGA).

5. The implantable medical material of claim 1, wherein said first fiber is manufactured from materials selected from among the group of materials consisting of Dacron, polyester (PET), Teflon and Polyethylene Naphthalate (PEN).

6. The implantable medical material of claim 1, said second fibers comprising fibers of at least two different absorption rates, said rates being differentiated by at least one of the group of absorption rate differentiators consisting of different denier sizes, braiding, twisting, plying, and coating of said fibers.

7. The implantable medical material of claim 1, wherein the size of said relatively smaller interstices is less than 0.2 mm in at least one dimension.

8. The implantable medical material of claim 1, wherein the size of said relatively larger interstices is at least 0.75 mm in at least one dimension.

9. The implantable medical material of claim 1, wherein permeability of said implantable medical material is less than 250 cubic feet per minute (CFM).

10. The implantable medical material of claim 1, further comprising a bio-compatible coating on said material that reduces the respective said rates of bio-absorption.

11. The implantable medical material of claim 1, said implantable medical material comprising a woven material, said first and second fibers being interspersed in at least one of warp and fill directions of said woven material.

12. The implantable medical material of claim 11, said woven material incorporating intersection locks of said first fibers at the free edges of said material.

13. An implantable woven medical material comprising:
a first pattern of multiple, relatively larger interstices defined by first fibers of relatively lower rates of bio-absorption;
a second pattern of multiple, relatively smaller interstices within said first pattern defined by second fibers of relatively higher rates of bio-absorption in combination with said first fibers;
a third pattern of multiple, relatively yet smaller interstices within said first pattern defined by third fibers of relatively yet higher rates of bio-absorption in combination with said first and said second fibers;
said first fibers comprising non-absorbable fibers,
said second and third fibers being made of Poly Glycolic Acid (PGA), and
said first, second and third fibers being interspersed in said woven medical material wherein the size of said relatively yet smaller interstices is less than 0.2 mm in at least one dimension, the size of said relatively larger interstices is at least 0.75 mm, and the permeability of said woven medical material is less than 250 cubic feet per minute (CFM).

14. A method for making an implantable medical material comprising:
combining first fibers of relatively lower rates of bio-absorption with second fibers of relatively higher rates of bio-absorption into an integrated pattern of multiple, relatively larger interstices defined by said first fibers and relatively smaller interstices within said relatively larger interstices, said smaller interstices defined by said first and second fibers.

15. The method of claim 14 for making an implantable medical material, further comprising;
combining said first and second fibers with third fibers of relatively yet higher rates of bio-absorption wherein said integrated pattern further includes relatively yet smaller interstices within said relatively larger interstices, said yet smaller interstices defined by said first, second and third fibers.

16. The method of claim 15 for making an implantable medical material, further comprising;
making said second and said third fibers from Poly Glycolic Acid (PGA).

17. The method of claim 16 for making an implantable medical material, further comprising;
making said first fiber from materials selected from among the group of materials consisting of Dacron, polyester (PET), Teflon and Polyethylene Naphthalate (PEN).

18. The method of claim 14 for making an implantable medical material, further comprising;
adjusting the rate of bio-absorption of said medical material by selecting and preparing at least some of said second fibers for said combining according to at least one criteria selected from among the group of criteria consisting of denier sizing, braiding, twisting, plying, and coating.

19. The method of claim 14 for making an implantable medical material, comprising said relatively smaller interstices being less than less than 0.2 mm in at least one dimension, and said relatively larger interstices being at least 0.75 mm in at least one dimension.

20. The method of claim 14 for making an implantable medical material, said material comprising a permeability of less than 250 cubic feet per minute (CFM) in accordance with the Frazier Permeability test.
